(19) United States
(12) Patent Application Publication
(10) Pub. No.: US 2020/0289392 A1
(43) Pub. Date: Sep. 17, 2020

(54) A MELANOGENESIS INHIBITOR COMPOSITION

(71) Applicants: Dow Global Technologies LLC, Midland, MI (US); Rohm and Haas Company, Collegeville, PA (US)

(72) Inventors: Kinjalbahen Joshi, Collegeville, PA (US); Yujing Tan, Midland, MI (US); Cassie Fhaner, Freeland, MI (US)

(73) Assignees: Dow Global Technologies LLC, Midland, MI (US); Rohm and Haas Company, Collegeville, PA (US); Rohm and Haas Company, Collegeville, PA (US)

(21) Appl. No.: 16/618,839

(22) PCT Filed: Jun. 6, 2018
(86) PCT No.: PCT/US2018/036217

§ 371 Date: Dec. 3, 2019

Related U.S. Application Data
(60) Provisional application No. 62/525,309, filed on Jun. 27, 2017.

(51) Int. Cl. A61K 8/49; A61K 8/40; A61Q 19/02

(52) U.S. Cl. CPC A61K 8/498; A61K 2800/10; A61Q 19/02; A61K 8/40

(57) ABSTRACT

A melanogenesis inhibitor combination is provided, comprising: a combination of (Z)—N-(4-hydroxybenzylidene)propan-2-amine oxide; and a compound of formula I

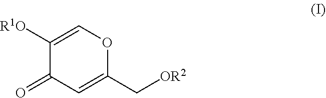

wherein R¹ and R² are independently selected from a hydrogen atom and an acyl group having 3 to 20 carbon atoms; wherein the combination is dispersed in a dermatologically acceptable carrier.

The present invention relates to melanogenesis inhibitors. In particular, the present invention relates to a melanogenesis inhibitor combination, comprising: a combination of (Z)—N-(4-hydroxybenzylidene)propan-2-amine oxide; and a compound of formula I; wherein R¹ and R² are independently selected from a hydrogen atom and an acyl group having 3 to 20 carbon atoms; wherein the combination is dispersed in a dermatologically acceptable carrier.

Melanin is the primary determinant of skin color in humans. Melanocytes in the epidermis produces melanin in the skin in response to certain environmental triggers (e.g., increased sun exposure).

There is an increasing interest in products in the field of cosmetics that effect depigmenting of, for example, age spots, chloasma, freckles, pigmentation spots that appear on the skin as individuals age sometimes aggravated by prolonged exposure to the sun or tanning lights. There is also an increased fashion phenomena favoring lighter complexions.

Oxidation reactions within the melanocytes result in the formation in melanin. These oxidation reactions may be catalyzed by tyrosinase. Resulting pigments are uniformly distributed over the epidermis, except when the mechanism is disrupted resulting in an accumulation of melanin in localized areas.

One well known substance with depigmenting action (melanogenesis inhibition) is kojic acid. However, there has been an interest in reducing the level of kojic acid in some formulations.

Accordingly, there remains a need for compositions having melanogenesis inhibition that facilitate a reduction in the amount of kojic acid used in skin whitening formulations.

The present invention provides a melanogenesis inhibitor combination, comprising: a combination of a (Z)—N-(4-hydroxybenzylidene)propan-2-amine oxide; and a compound of formula I

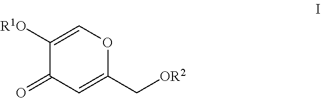

wherein R¹ and R² are independently selected from a hydrogen atom and an acyl group having 3 to 20 carbon atoms; wherein the combination is dispersed in a dermatologically acceptable carrier.

The present invention provides a melanogenesis inhibitor combination, comprising: a combination of a (Z)—N-(4-hydroxybenzylidene)propan-2-amine oxide; and a compound of formula I selected from the group consisting of kojic acid, kojic acid monobutyrate, kojic acid monocaprate, kojic acid monopalmitate, kojic acid monostearate, kojic acid monocinnamoate, kojic acid monobenzoate, kojic acid dibutyrate, kojic acid dipalmitate, kojic acid distearate and kojic acid dioleate; wherein the composition is dispersed in a dermatologically acceptable carrier.

The present invention provides a melanogenesis inhibitor combination, comprising: a combination of a (Z)—N-(4-hydroxybenzylidene)propan-2-amine oxide; and a compound of formula I selected from the group consisting of kojic acid, kojic acid monobutyrate, kojic acid monocaprate, kojic acid monopalmitate, kojic acid monostearate, kojic acid monocinnamoate, kojic acid monobenzoate, kojic acid dibutyrate, kojic acid dipalmitate, kojic acid distearate and kojic acid dioleate; wherein the composition is dispersed in a dermatologically acceptable carrier selected from the group consisting of at least one of an emulsion, a cream, an aqueous solution, an oil, an ointment, a paste, a gel, a lotion, a milk, a foam, a suspension and a powder.

The present invention provides a method of inhibiting melanogenesis activity and melanin production in an animal having skin to improve appearance of the skin, comprising topically applying a melanogenesis inhibitor combination of the present invention to the skin.

### DETAILED DESCRIPTION

We have surprisingly found that pHBz-IPHA has significant melanogenesis inhibition activity particularly when paired with a compound of formula I.

Unless otherwise indicated, ratios, percentages, parts, and the like are by weight.

The term “cosmetically acceptable” as used herein and in the appended refers to ingredients typically used in personal care compositions, and is intended to underscore that materials that are toxic when present in the amounts typically found in personal care compositions are not contemplated as part of the present invention.

Preferably, the melanogenesis inhibitor combination of the present invention, comprises: a combination (preferably, 0.0001 to 5 wt %; more preferably, 0.0001 to <0.01; most preferably, 0.001 to <0.01 wt %) of a (Z)—N-(4-hydroxybenzylidene)propan-2-amine oxide (pHBz-IPHA); and (preferably, 0.05 to 5 wt %; more preferably, 0.1 to 2.5 wt %; most preferably, 0.5 to 2 wt %) of a compound of formula I

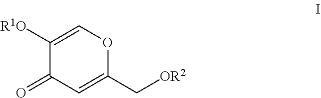

wherein R¹ and R² are independently selected from a hydrogen atom and an acyl group having 3 to 20 carbon atoms; wherein the combination is dispersed in a dermatologically acceptable carrier.

Preferably, the melanogenesis inhibitor combination of the present invention, comprises a combination including (Z)—N-(4-hydroxybenzylidene)propan-2-amine oxide (pHBz-IPHA) having the following structure

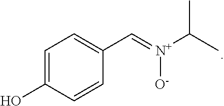

Preferably, the melanogenesis inhibitor composition of the present invention, comprises a combination including 0.0001 to 5 wt % of a pHBz-IPHA. More preferably, the melanogenesis inhibitor composition of the present invention, comprises a combination including 0.0001 to <0.01 wt % of a pHBz-IPHA. Most preferably, the melanogenesis inhibitor composition of the present invention, comprises a combination including 0.001 to <0.01 wt % of a pHBz-IPHA.

(Z)—N-(4-hydroxybenzylidene)propan-2-amine oxide (pHBz-IPHA) may be readily prepared by those skilled in the art using known synthetic techniques.

Preferably, the melanogenesis inhibitor combination of the present invention, comprises a combination including a compound of formula I

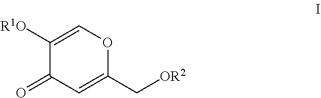

wherein R¹ and R² are independently selected from a hydrogen atom and an acyl group having 3 to 20 carbon atoms. More preferably, the melanogenesis inhibitor combination of the present invention, comprises a combination including 0.05 to 5 wt % of a compound of formula I; wherein R¹ and R² are independently selected from a hydrogen atom and an acyl group having 3 to 20 carbon atoms. Still more preferably, the melanogenesis inhibitor combination of the present invention, comprises a combination including 0.1 to 2.5 wt % of a compound of formula I; wherein R¹ and R² are independently selected from a hydrogen atom and an acyl group having 3 to 20 carbon atoms. Most preferably, the melanogenesis inhibitor combination of the present invention, comprises a combination including 0.5 to 2 wt % of a compound of formula I; wherein R¹ and R² are independently selected from a hydrogen atom and an acyl group having 3 to 20 carbon atoms (preferably, wherein R¹ is an —OH group).

Preferably, the melanogenesis inhibitor combination of the present invention, comprises a combination including a compound of formula I

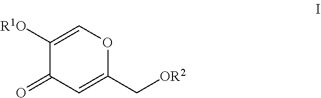

wherein the compound of formula I is selected from the group consisting of kojic acid, kojic acid monobutyrate, kojic acid monocaprate, kojic acid monopalmitate, kojic acid monostearate, kojic acid monocinnamoate, kojic acid monobenzoate, kojic acid dibutyrate, kojic acid dipalmitate, kojic acid distearate and kojic acid dioleate.

Preferably, the melanogenesis inhibitor combination of the present invention, comprises a combination including a compound of formula I

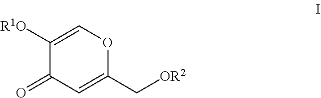

wherein the compound of formula I is selected from the group consisting of kojic acid, kojic acid monobutyrate, kojic acid monocaprate, kojic acid monopalmitate, kojic acid monostearate, kojic acid monocinnamoate, kojic acid monobenzoate, kojic acid dibutyrate, kojic acid dipalmitate, kojic acid distearate and kojic acid dioleate; and wherein R¹ is an —OH group.

Preferably, the melanogenesis inhibitor combination of the present invention, comprises a combination including a compound of formula I, wherein the compound of formula I includes Kojic acid. More preferably, the melanogenesis inhibitor combination of the present invention, comprises a combination including a compound of formula I, wherein the compound of formula I is Kojic acid.

Preferably, the melanogenesis inhibitor combination of the present invention, comprises a dermatologically acceptable carrier; wherein the combination is dispersed in the dermatologically acceptable carrier. More preferably, the melanogenesis inhibitor combination of the present invention, comprises 50 to 99.99 wt % of a dermatologically acceptable carrier; wherein the combination is dispersed in the dermatologically acceptable carrier.

Preferably, the dermatologically acceptable carrier is selected from the group consisting of at least one of an emulsion, a cream, an aqueous solution, an oil, an ointment, a paste, a gel, a lotion, a milk, a foam, a suspension and a powder. The dermatologically acceptable carrier is typically characterized as a vehicle or a diluent that does not cause significant irritation to the skin and does not negate the melanogenesis inhibitor activity of the combination in the composition. The dermatologically acceptable carrier used in the present invention may include water, a thickener, an emollient, an emulsifier, a humectant, a surfactant, a suspending agent, a film forming agent, a foam building agent, a preservative, an antifoaming agent, a fragrance, a lower monoalcoholic polyol, a high boiling point solvent, a propellant, a colorant, a pigment, glycerin, a mineral oil, silicon feel modifiers, preservatives, emollients and mixtures thereof.

The melanogenesis inhibitor combination of the present invention may be used in a variety of personal care applications including, for example, cosmetics and in skin care (e.g., lotions, creams, oils, topical medicines).

Preferably, the method of inhibiting melanogenesis activity and melanin production in an animal having skin to improve appearance of the skin of the present invention comprises topically applying a melanogenesis inhibitor combination of the present invention to the skin. More preferably, the method of inhibiting melanogenesis activity and melanin production in an animal having skin to improve appearance of the skin of the present invention comprises topically applying a melanogenesis inhibitor combination of the present invention to the skin; wherein the melanogenesis inhibitor combination topically applied to the skin contains 0.001 to <0.01 wt % of pHBz-IPHA. Most preferably, the method of inhibiting melanogenesis activity and melanin production in an animal having skin to improve appearance of the skin of the present invention comprises topically applying a melanogenesis inhibitor combination of the present invention to the skin; wherein the melanogenesis inhibitor combination topically applied to the skin contains 0.001 to <0.01 wt % of pHBz-IPHA and kojic acid.

The melanogenesis inhibitor combination of the present invention can be manufacture using processes well know in the art, e.g., by means of conventional mixing, dissolving, granulating, emulsifying, encapsulating, entrapping and lyophilizing processes.

Some embodiments of the present invention will now be described in detail in the following Examples.

Some embodiments of the present invention will now be described in detail in the following Examples.

### Synthesis of VAN-IPHA

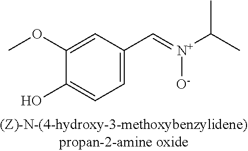

A 100 mL 1-neck flask was equipped with a magnetic stirrer and a rubber septum. The flask was charged with (3.80 g; ˜0.025 moles) of vanillin, (1.88 g; ˜0.025 moles) of isopropylhydroxyamine, and 25 mL of methanol. The solution was stirred at room temperature over the weekend, during which time the nitrone separated as white crystals. The product nitrone, (Z)—N-(4-hydroxy-3-methoxybenzylidine)propan-2-amine oxide (VAN-IPHA), was isolated by filtration. The product was then washed on the filter with a small amount of methanol and was air dried.

### Synthesis of pHBz-IPHA

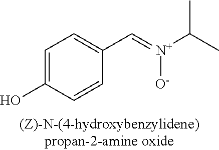

A 100 mL 1-neck flask was equipped with a magnetic stirrer and a rubber septum. The flask was charged with (3.05 g; ˜0.025 moles) of p-hydroxybenzaldehyde, (1.88 g; ˜0.025 moles) of isopropylhydroxylamine, and 25 mL of methanol. The solution was stirred at room temperature over the weekend, during which time the nitrone separated as white crystals. The product nitrone, (Z)—N-(4-hydroxybenzylidene)propan-2-amine oxide oxide (pHBz-IPHA), was isolated by filtration. The product was then washed on the filter with a small amount of methanol and was air dried.

### Tyrosinase Inhibition

The tyrosinase inhibition activity of VAN-IPHA and pHBz-IPHA at 0.1 wt % active concentration was compared to the activity exhibited by 0.1 wt % kojic acid. The VAN-IPHA showed negligible tyrosinase inhibition activity. The pHBz-IPHA showed about 60% tyrosinase inhibition activity compared to that of kojic acid.

### COMPARATIVE EXAMPLES C1-C6 and EXAMPLES 1-2

### In Vitro Melanogenesis Inhibition

Skin depigmentation activity for the formulations of Comparative Examples C1-C6 and Examples 1-2, as described in TABLE 1, were tested in triplicate using in vitro co-culture differentiated epidermal skin model (MelanoDerm™ tissue model MEL-300-B available from MatTek Corporation).

### Day 1: Initial Tissue Sample Conditioning

On day 1, eight separate 4×6-well sterile plates were filled with 900 μL of an assay medium (EPI-100NMM-113 available from Kurabo Corporation). Using sterile forceps three skin tissues (MelanoDerm™ tissue model number MEL-300-B available from MatTek Corporation) were transferred to the top row of each one of the 4×6-well sterile plates prefilled with the assay medium. The wells were then incubated under a 5% CO₂ atmosphere at 37° C. for 16 hours.

### Day 2: Initial Tissue Sample Treatment

On day 2, the well plates were removed from the incubator and 5 mL of the assay medium was added to each of the eight separate 4×6-well sterile plates. Each of the three skin tissues in the top row of each of the well plates was then topically dosed with 25 μL of one of the formulations of Comparative Examples C1-C6 and Examples 1-2. After dosing the well plates were then incubated under a 5% CO₂ atmosphere at 37° C. for 48 hours.

### Subsequent Tissue Sample Wash and Treatment

On each of days 4, 6, 8, 10 and 12; the well plates were removed from the incubator and the three skin tissues in the top row of each of the well plates was then washed with sterile 1× Dulbecco's phosphate buffered saline (1× DPBS). The sterile 1× DPBS was then aspirated off the skin tissues, which were then blot dried using a sterile paper towel. Then each of the three skin tissues in the top row of each of the well plates was then topically dosed with 25 μL of one of the formulations of Comparative Examples C1-C6 and Examples 1-2. After dosing the well plates were then incubated under a 5% CO₂ atmosphere at 37° C. for 48 hours.

### Day 14: Melanin Assay

On day 14; the well plates were removed from the incubator and the three skin tissues in the top row of each of the well plates was then washed with sterile 1× DPBS. The sterile 1× DPBS was then aspirated off the skin tissues, which were then blot dried using a sterile paper towel. A melanin colorimetric assay was used to determine the melanin content of each of the three skin tissues in the top row of each of the well plates by calculating the melanin concentration of the tissue inserts against synthetic melanin solution (available from Sigma-Aldrich). The assays were performed by taking tissue extracts using an aqueous based solubilizer (Solvable™ tissue solubilizer available from PerkinElmer), incubating overnight at 60° C. and then measuring the absorbance at 490 nm with a 96-well plate reader. Then averages for each formulation are reported in TABLE 1.

TABLE 1

| | | | | | Melanin |
|---|---|---|---|---|---|
| | kojic Acid¹ | VAN-IPHA | pHBz-IPHA | Solvable² | Content |
| Ex. | (wt %) | (wt %) | (wt %) | (wt %) | (μg/tissue) |
| C1 | — | — | — | 100 | 13.4 |
| C2 | 2 | — | — | 98 | 6.3 |
| C3 | — | 0.1 | — | 99.9 | 10.7 |
| C4 | 2 | 0.05 | — | 97.95 | 5.7 |
| C5 | 2 | 0.01 | — | 97.99 | 6.2 |
| C6 | — | — | 0.1 | 99.9 | 9.4 |
| 1 | 2 | — | 0.05 | 97.95 | 6.5 |
| 2 | 2 | — | 0.01 | 97.99 | 5.6 |

¹Kojic Acid available from Sigma-Aldrich, Co.
²Solvable ™ aqueous based solubilizer available from PerkinElmer

We claim:

1. A melanogenesis inhibitor combination, comprising:
a combination of
a (Z)—N-(4-hydroxybenzylidene)propan-2-amine oxide; and
a compound of formula I 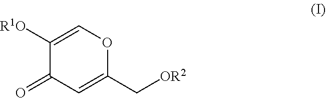
wherein R¹ and R² are independently selected from a hydrogen atom and an acyl group having 3 to 20 carbon atoms;
wherein the combination is dispersed in a dermatologically acceptable carrier.

2. The melanogenesis inhibitor combination of claim 1, wherein the compound of formula I is selected from the group consisting of kojic acid, kojic acid monobutyrate, kojic acid monocaprate, kojic acid monopalmitate, kojic acid monostearate, kojic acid monocinnamoate, kojic acid monobenzoate, kojic acid dibutyrate, kojic acid dipalmitate, kojic acid distearate, kojic acid dioleate.

3. The melanogenesis inhibitor combination of claim 2, wherein R¹ is an —OH group.

4. The melanogenesis inhibitor combination of claim 3, wherein the combination contains 0.001 to <0.01 wt % of pHBz-IPHA.

5. The melanogenesis inhibitor combination of claim 1, wherein the compound of formula I is kojic acid.

6. The melanogenesis inhibitor combination of claim 4, wherein the compound of formula I is kojic acid.

7. The melanogenesis inhibitor combination of claim 1, wherein the dermatologically acceptable carrier is selected from the group consisting of at least one of an emulsion, a cream, an aqueous solution, an oil, an ointment, a paste, a gel, a lotion, a milk, a foam, a suspension and a powder.

8. A method of inhibiting melanogenesis activity and melanin production in an animal having skin to improve appearance of the skin, comprising topically applying a melanogenesis inhibitor combination of claim 1 to the skin.

9. The method of claim 8, wherein the melanogenesis inhibitor combination topically applied to the skin contains 0.001 to <0.01 wt % of pHBz-IPHA.

10. The method of claim 8, wherein the melanogenesis inhibitor combination topically applied to the skin contains 0.001 to <0.01 wt % of pHBz-IPHA and kojic acid.
